(19) United States
(12) Patent Application Publication
(10) Pub. No.: US 2022/0183637 A1
(43) Pub. Date: Jun. 16, 2022

(54) METHOD FOR DETERMINING AN ADAPTATION OF AN IMAGING EXAMINATION

(71) Applicant: Siemens Healthcare GmbH, Erlangen (DE)

(72) Inventors: Jens Thöne, Lauf an der Pegnitz (DE); Elisa Marchetta, Erlangen (DE)

(73) Assignee: Siemens Healthcare GmbH, Erlangen (DE)

(21) Appl. No.: 17/553,395

(22) Filed: Dec. 16, 2021

(30) Foreign Application Priority Data
Dec. 16, 2020 (DE) 102020216040.6

(51) Int. Cl. A61B 5/00; G10L 15/22; G10L 15/16; G10L 15/14; G16H 30/40; G06N 3/02

(52) U.S. Cl. CPC A61B 5/749; G10L 15/22; G10L 15/16; A61B 5/055; G16H 30/40; G06N 3/02; G10L 2015/223; G10L 15/14

(57) ABSTRACT

A computer-implemented method for determining an adaptation of a parameter of an imaging examination that is to be carried out using a medical imaging apparatus in dependence on an input of information for the imaging examination, including: capturing the information input for the imaging examination; determining an item of information relating to an adaptation of the imaging examination in dependence on the information input; allocating the item of information relating to the adaptation of the imaging examination to a parameter of the imaging examination; determining an adaptation of the parameter of the imaging examination in dependence on the information input; and providing a parameter set of the imaging examination.

### TECHNICAL FIELD

The disclosure relates to a computer-implemented method for determining an adaptation of a parameter of an imaging examination that is to be carried out using a medical imaging apparatus in dependence on an input of information for the imaging examination. Further, the disclosure relates to an imaging apparatus, comprising a computing unit and a computer program product that are formed to perform a method according to the disclosure.

### BACKGROUND

In diagnostic imaging for capturing images of a patient by means of an imaging apparatus, imaging sequences are typically used. These imaging sequences may comprise a plurality of imaging parameters that establish for example a workflow of an imaging examination, or a quality of the images.

Conventionally, imaging sequences are created and parameterized independently of the imaging examination. The quality of the images may be crucially dependent on specific preconditions in the patient, which can only be taken into account to a limited extent during creation of the imaging sequences. For this reason, a user of the imaging apparatus is frequently instructed to adapt the imaging parameters manually to preconditions of the patient, using an appropriate editor, during the imaging examination. Frequent changes, for example reflecting a particular preference of the user in respect of imaging, or taking into account a specific region of the patient's body, are noted by the user as they occur and are subsequently transferred manually to stored imaging sequences (e.g. standard sequences). Typically, transmitting changed imaging parameters to the imaging apparatus during an imaging examination requires manual coordination by the user, since changes to the imaging parameters made in the editor are not transferred directly to the imaging apparatus. These constraints apply likewise to adaptation of a workflow of the imaging examination by the user. Conventionally, a user who has had medical and technical training is required for adapting the imaging examination, and such a user is not always available.

### SUMMARY

It is an object of the disclosure to enable an adaptation of a parameter of an imaging examination to be determined in a simplified manner.

The computer-implemented method according to the disclosure for determining an adaptation of a parameter of an imaging examination that is to be carried out using a medical imaging apparatus in dependence on an input of information for the imaging examination has the following steps:
capturing the information input for the imaging examination,
determining an item of information relating to an adaptation of the imaging examination in dependence on the information input,
allocating the item of information relating to the adaptation of the imaging examination to a parameter of the imaging examination,
determining an adaptation of the parameter of the imaging examination in dependence on the information input, and
providing a parameter set of the imaging examination, wherein the provided parameter set has a parameter that is changed in accordance with the determined adaptation, and wherein the parameter set is stored in a storage unit that is connected to an imaging apparatus.

An imaging examination may be an examination of a patient by imaging for the purpose of capturing images of a region of the body that is diagnostically relevant. The method relates in particular to an imaging examination that is to be carried out. This may mean that the imaging examination is already in planning and/or preparation. It is likewise conceivable that the imaging examination is to be carried out within a foreseeable period, for example in at most a day's time, at most an hour's time, at most half an hour's time or a few minutes' time. It is furthermore conceivable that the imaging examination has already started at the time of carrying out the method according to the disclosure.

The imaging examination may in particular comprise one or more imaging parameters and/or one or more imaging sequences that are directed to capturing images of the region of the body that is diagnostically relevant. The imaging examination is preferably carried out by means of a medical imaging apparatus. A medical imaging apparatus may be any desired medical device that is formed to capture two-dimensional or three-dimensional image data of the patient. Examples of such imaging devices are magnetic resonance tomography devices, computed tomography devices, X-ray devices, mammography devices, positron emission tomography devices, single-photon emission computed tomography devices, ultrasound devices and similar. An image that has been captured by means of the imaging apparatus may comprise a two-dimensional or three-dimensional representation of a region of the patient's body. In a preferred embodiment, the imaging apparatus is a magnetic resonance imaging apparatus and the imaging examination is a magnetic resonance imaging examination. In particular, the imaging apparatus has a computing unit that is formed to coordinate the method according to the disclosure and to carry it out by means of the imaging apparatus. It is likewise conceivable that the method according to the disclosure is carried out by means of a controller of the imaging apparatus.

A parameter of an imaging examination that is to be carried out may be an imaging parameter such as an image resolution, a contrast, a signal-to-noise ratio, a specific absorption rate, a time to echo, a repetition time or similar. It is likewise conceivable that the parameter comprises a group of imaging parameters, an imaging sequence and/or a series of imaging sequences. Furthermore, a parameter may comprise any desired adjustment of the imaging examination and/or a workflow of the imaging examination.

An information input for the imaging examination preferably comprises a signal that has and/or carries an item of information on the imaging examination and/or the workflow of the imaging examination. In particular, the information input may refer to an imaging parameter of the imaging examination, an imaging sequence, a series of imaging sequences and/or any desired adjustment that relates to a workflow of the imaging examination. The information input may be transmitted to the imaging apparatus in the form of an analog or digital signal. However, it is likewise conceivable that the information input comprises an acoustic signal and/or an optical signal. The imaging apparatus preferably has a suitable interface and/or a suitable sensor that are intended to capture the information input. The term “capturing the information input” may in particular mean that a signal from a user of the imaging apparatus is received by means of an interface and/or a sensor. It is furthermore conceivable that capture of the information input comprises conversion of the received signal into machine-readable data. For example, the information input may be an input by the user at a keyboard, a mouse, a touch panel, or similar. However, it is likewise conceivable that the information input comprises a speech message and/or a gesture by the user, which are captured by means of a sound converter such as a microphone or a sound sensor, and/or an optical sensor such as a 2D camera, 3D camera or infrared camera.

Determining an item of information relating to an adaptation of the imaging examination, in dependence on the information input, may comprise an analysis of the information input in respect of a change that is to be carried out to a parameter of the imaging examination. In particular here, the information input may be checked for correlation with an imaging parameter, an imaging sequence and/or an adjustment of the workflow of the imaging examination. Here, a search and/or analysis, such as a semantic analysis, may for example be carried out by means of an artificial neural network and/or a model-based approach. It is furthermore conceivable that the item of information relating to the adaptation of the imaging examination comprises an indication of a direction in which the adaptation of a parameter is to be made. An indication of this kind may be captured with the information input or be derived in the case of determining the item of information relating to the adaptation of the imaging examination in dependence on the information input.

For example, the information input may take the form of an acoustic signal that comprises an item of information from the user on a desired change to an image property. The acoustic signal may comprise a voiced designation of an imaging parameter and/or an image property. In this case, determining the item of information relating to the adaptation of the imaging examination may comprise speech processing. In a further example, the information input may take the form of an optical signal, such as a gesture by the user, which encodes an image property and/or an imaging parameter. In this case, determining the item of information relating to the adaptation of the imaging examination may comprise image processing. It is likewise conceivable that the information input comprises an electrical signal or a sequence of electrical signals that carry an item of information relating to a desired change to the image property in the form of machine-readable data. The machine-readable data may in this case be in any desired file format. The imaging apparatus may be formed to receive the information input and extract the information relating to the adaptation of the imaging examination. In a preferred embodiment, the information input is a speech input of the user that comprises an instruction on a change to an image property. For the purpose of receiving the speech input of the user and extracting the desired change to the image property, a speech input unit and/or a speech processing unit may be used. Similarly, an image processing unit and/or a computing unit may be used to process for example a gesture by the user, using image data of an optical sensor or a keyboard input by the user.

Allocating the item of information relating to the adaptation of the imaging examination to a parameter of the imaging examination may be performed for example by way of a classification. In the classification, the item of information on the change to the imaging examination may be allocated to a parameter of the imaging examination. Preferably, an artificial neural network, a multilayered neural network and/or a text mining method are used to classify the item of information relating to the change to the imaging examination. The classification may comprise formation of a tuple, a vector, a matrix and/or a data structure, which allocate the item of information relating to the change to the imaging examination to a parameter of the imaging examination. It is furthermore conceivable that allocation of the item of information relating to the adaptation of the imaging examination is performed using a model, such as a statistical model and/or a logical data model.

Determining the adaptation of the parameter of the imaging examination, in dependence on the information input, may comprise establishing a change in a value of a parameter or a plurality of parameters. Establishing the change in the value of a parameter is in this case performed in particular in dependence on the item of information relating to the adaptation of the imaging examination. It is conceivable that an adapted parameter first comprises a proposal to adapt a parameter, wherein the proposal takes into account the information relating to adaptation of the imaging examination. The proposal may for example be output to the user, who confirms the proposal before adaptation of a parameter of the imaging examination is put into effect. However, it is likewise conceivable that the adaptation of the parameter is implemented automatically within the context of the method according to the disclosure in order to carry out the imaging examination with the adapted parameter.

Determining the adaptation of the parameter may be carried out in dependence on the indication of the direction in which the adaptation of the parameter is to be made. Preferably, determining the adaptation of the parameter comprises establishing a concrete value that corresponds to the indication of the direction of the adaptation. Adaptation of the parameter is determined at least in dependence on an information input. However, it is likewise conceivable that adaptation of the parameter is determined in dependence on a plurality of information inputs, such as the imaging examination, an item of information on the patient, a region of the body to be examined, a booking of the imaging apparatus, a standard setting of the imaging examination or similar. In one example, the item of information relating to the adaptation of the imaging examination may comprise a desire of the user for a higher resolution and/or an enlarged field of view. In this case, concretely establishing the imaging parameters concerned is preferably performed taking into account an information input on the size and/or weight of a patient. This makes it possible to avoid impermissibly exceeding a specific absorption rate and/or duration of the imaging examination. Establishing the concrete value of the parameter may furthermore be performed in predetermined increments and/or using an optimizer that takes into account a plurality of information inputs.

Providing a parameter set may also comprise outputting and/or carrying out an imaging examination. The parameter set for imaging examination may comprise a plurality of imaging parameters, imaging sequences and/or further parameters that are characteristic of an imaging examination of the region of the patient's body that is diagnostically relevant. It is conceivable that a parameter is changed in accordance with the determined adaptation and is stored, with a parameter set, in a storage unit. The parameter set with the changed parameter may in this case be used in particular for a subsequent imaging examination. Further, the parameter set with the changed parameter may be output to a display unit and/or a further imaging apparatus. The further imaging apparatus may in this case be located in the same network and/or the same clinical facility in which the imaging apparatus is also located. The further imaging apparatus may in particular have the same measuring principle. As a result of providing the parameter set with the changed parameter, a changed imaging examination may advantageously also be applied on a further imaging apparatus. Furthermore, the user may make changes to the parameter set as they are output to a display unit in a particularly efficient way, and deliver feedback thereon. In this case, the parameter set with the changed parameter may in particular be a proposal, made to the user, of a possible adaptation of the parameter. The user is thus in a position to accept or reject the proposal for the imaging examination to be carried out.

As a result of providing the method according to the disclosure, it is advantageously possible to achieve time-efficient adjustment of parameters of the imaging examination. Further, the imaging examination may advantageously be adapted to specific requirements of the user and/or the patient, which in the case of a conventional setting of parameters is not practicable because of the number of steps or actions required.

In one embodiment of the method according to the disclosure, the parameter of the imaging examination comprises an imaging parameter, an imaging sequence and/or a parameter of a workflow of the imaging examination.

An imaging parameter may comprise for example a resolution, an imaging region or an imaging duration. Examples of parameters of a magnetic resonance examination are a time to echo, a repetition time, a k-space cover, a specific absorption rate or similar. An imaging sequence may comprise in particular a temporal series of steps and/or parameters of image data capture. For example, an imaging sequence may have a temporal series of excitation intervals, measurement intervals, breaks, respiration intervals and/or instructions for the patient. It is likewise conceivable that the imaging sequence comprises a plurality of successive imaging sequences. A parameter of a workflow of the imaging examination may be any desired setting relating to a preparation and/or the carrying out of the imaging examination. Parameters of this kind may comprise for example a movement of the patient table, a position of the patient relative to the imaging apparatus, a positioning of a local coil, capture of optical image data, in particular capture of optically active markers and/or magnetic resonance-active markers for adjusting a position of the patient table and/or a local coil, capture of a navigator measurement, such as a projection image and/or a scout image measurement.

Adapting an imaging parameter advantageously allows image properties of a captured image to be adapted to a desire of the user in an efficient and simple manner. Further, by means of the item of information relating to the adaptation of the imaging examination, it is advantageously possible to adapt a plurality or a group of parameters, as a result of which efficiency of the imaging examination and/or of the preparation of the imaging examination is enhanced. For example, by means of an item of information on a reduction of the imaging examination, both imaging parameters and parameters of the workflow of the imaging examination can be adapted.

In a preferred embodiment of the method according to the disclosure, the information input comprises a speech input from a user, wherein capture of the information input comprises processing of the speech input from the user, and wherein adaptation of the parameter is determined in dependence on the speech input from the user.

A speech input from the user may be captured using any desired speech input unit. Preferably, the speech input from the user is converted into machine-readable data by means of the speech input unit. Then, the speech input can be processed by means of a computing unit and/or a dedicated speech processing unit. It is conceivable that the speech input is processed using a pipeline model and/or a semantic network, for example an artificial neural network, a multilayered neutral network (deep learning), in particular a MultiNet (multilayered extended semantic networks). The speech processing may have one or more of the following steps:
speech recognition,
tokenization,
morphological analysis,
syntactical analysis,
semantic analysis, and
dialog analysis.

In one embodiment, the speech input from the user is processed by means of an artificial neural network or a multilayered neural network. However, it is likewise conceivable for one or more of the listed steps of speech processing to be performed using an artificial neural network or a multilayered neural network. Artificial neural networks and multilayered neural networks may advantageously be trained using large quantities of data in order to robustly and reproducibly process even linguistically complex and/or colloquial speech inputs from the user. In particular, artificial neural networks or multilayered neural networks may advantageously be trained to identify speech from the user that lacks clarity, such as the misnaming of an imaging parameter or a change in the designation of an image property.

According to one embodiment, the speech input from the user is processed by means of a statistical model and/or a logical data model. Models of this kind may be incorporated into a method for processing the speech input using the pipeline model and/or may carry out or support individual or all of the above-listed steps. It is conceivable that, when corresponding models are used, the user makes use of a predetermined selection of terms and/or instructions, wherein this selection is matched to the imaging apparatus and/or the speech input unit. The predetermined selection of terms and/or instructions may comprise a restricted vocabulary that covers for example some of the parameters of the imaging examination and possible adaptation options of the parameters. As a result of using statistical and/or logical data models, the restricted vocabulary may advantageously be captured and processed without much work. Preferably, an item of information relating to adaptation of the imaging examination, in particular of a parameter of the imaging examination, is determined from the speech input from the user by means of one or more of the methods described above for processing speech input.

As a result of a possibility provided by the speech input, a request to adapt a parameter can be transmitted to the imaging apparatus particularly time-efficiently. In the case of a magnetic resonance examination or computed tomography examination, it may be that when the speech input is delivered the user is in an examination room and not at a user interface of the imaging apparatus, which is conventionally located in a separate room. As a result, the user can deliver a speech input for adapting a parameter of the imaging examination in parallel with preparing the patient for the imaging examination, as a result of which efficiency of the preparation of the imaging examination can advantageously be enhanced.

According to one embodiment, the method according to the disclosure has the further step of:
outputting a captured image of the imaging examination to the user of the imaging apparatus,
wherein capture of the information input comprises capturing feedback from the user on the captured image of the imaging examination, wherein adaptation of the parameter is determined in dependence on the feedback from the user.

It is conceivable that the imaging examination comprises a plurality of successive imaging sequences during which images of the patient are captured. Preferably, a first image is output to the user in order to obtain feedback from the user on the first image. The first image may be output to the user at any desired display unit, such as a screen of a user interface or a display of a mobile device. The output may comprise a request for delivery of feedback. For example, the first image may be output together with an acoustic or text request—such as a speech message, a multiple-choice form, a chatbot or similar. Depending on the request, the user may give corresponding feedback on the first image.

In one embodiment, the imaging examination is adapted to the first image in dependence on the feedback from the user. Capture of the feedback from the user may in this case comprise an iterative procedure. For example, a first adaptation of a parameter does not yet correspond to a request by the user. A second image can then be output that is captured using an adapted parameter. At this stage, a second feedback message from the user can be obtained in order to further improve adaptation of the parameter. It is furthermore conceivable that the first image comprises a projection image or a scout image. In this case, the first image can be used to obtain feedback on an imaging region of the imaging examination from the user. In a further embodiment, the imaging examination comprises a plurality of imaging sequences. It is conceivable that in each case at least one first image of one or more of the plurality of imaging sequences is output to the user in order to obtain feedback from the user on the one or more imaging sequences.

As a result of capturing the feedback from the user on a captured image of the imaging examination, it can be ensured that the quality of the image of the imaging examination corresponds to a request by the user. This enables time-consuming repetition of the imaging examination to be avoided and/or a risk of misdiagnosis, because a finding is based on a low-quality image, to be advantageously avoided.

In a further embodiment of the method according to the disclosure, capture of the information input comprises capturing feedback from the user on an imaging parameter and/or an image property.

It is conceivable that the user of the imaging apparatus has expert knowledge on carrying out the imaging examination. In a case of this kind, the user may use the feedback to give a concrete instruction on adapting a parameter in dependence on the first image. The instruction may for example comprise a concrete proposal on adapting a resolution, a signal-to-noise ratio, a contrast, a series of imaging sequences, or other parameters. However, it is likewise conceivable that the user of the imaging apparatus is inexperienced in working with the imaging apparatus. In this case, the feedback from the user may comprise an indication of adaptation of a general image property rather than a concrete imaging parameter. For example, the feedback may comprise a speech input by the user such as “the image is not sharp enough”, “the image is noisy”, “the image is too dark” or “the image is too light”. It is likewise conceivable for the user to input a corresponding information input by way of a graphical user interface and/or a chatbot.

As a result of the possibility of capturing feedback on an image property, it is also possible to adapt the imaging examination in dependence on colloquially formulated feedback from users with relatively little specialist knowledge. Further, using the method according to the disclosure it is advantageously possible to enable an adaptation of imaging parameters in a particularly time-efficient manner, while inexperienced users are advantageously supported in the adaptation of parameters.

In a further embodiment of the method according to the disclosure, the information input comprises at least:
an input from the user,
a speech input from the user,
a feedback message from the user on a captured image of the imaging examination,
an item of information on a patient,
a clinical finding, and/or
an item of information on an image property.

An input from the user preferably comprises an adjustment to a parameter using a graphical user panel of a user interface of the imaging apparatus. Further, the input from the user may also be a gestural instruction. The speech input and feedback message from the user may take a form in accordance with an above-mentioned embodiment. An item of information on a patient may comprise any desired information on the patient, such as weight, age, gender, medical history, diagnostically relevant region of the body, mental state and/or mental health or similar. An item of information on a patient may in particular be a relevant constraint on determining the adaptation of the parameter. For example, a nervous state and/or claustrophobia in the patient may be taken into account when adapting a parameter that is directly or indirectly correlated with the duration of an imaging examination. It is likewise conceivable that a diagnostically relevant region of the patient's body is taken into account when determining an adaptation of the imaging region. In the case of a magnetic resonance examination, the weight of a patient may be taken into account when adjusting a parameter that is directly or indirectly correlated with the specific absorption rate. Preferably, an item of information on a patient is automatically entered, during the preparation or at the start of the imaging examination, from a hospital information system, a radiological information system, a patient file, and/or an internal or external data memory of a computing unit of the imaging apparatus. However, it is likewise conceivable that the item of information on the patient is manually entered by a user of the imaging apparatus, using a user interface of the imaging apparatus.

Similarly, a clinical finding may be taken into account when determining adaptation of the parameter. A clinical finding may in particular comprise a discovery of a pathological structure, in dependence on the first image or a subsequent image of the imaging examination. For example, the discovery of a tumor, aneurysm, bone fracture, thrombosis or similar may be an information input that triggers automatic determination of adaptation of a parameter. The information input may thus also be independent of an input by a user. In addition to an embodiment as described above, an image property may also comprise the presence of an image artifact such as a ghosting effect and/or smearing effect, and/or a signal-to-noise ratio. It is conceivable that image properties during capture of the first image or subsequent images are automatically analyzed in order to derive an item of information on the image property. The results of an analysis of this kind may be an information input that can be used in determining the adaptation of the parameter.

As a result of determining the adaptation of the parameter in dependence on an item of information on a patient, a clinical finding and/or an item of information on an image property, it is advantageously possible to enhance the quality of captured images. Further, the imaging examination may advantageously be automatically adapted to individual prerequisites of the patient.

In one embodiment, the method according to the disclosure has the following step:

training an intelligent algorithm in respect of determining an adaptation of a parameter, wherein the intelligent algorithm is trained at least in dependence on the information input, the imaging examination to be carried out, and the determined adaptation of the parameter.

An intelligent algorithm is preferably an artificial neural network, a multilayered neural network, an expert system and/or an optimization method. Preferably, training of the intelligent algorithm comprises at least a modification to a decision tree, a scalar, a cost function, an input variable, an output variable and/or a configuration of an artificial or neural network.

In a preferred embodiment, the intelligent algorithm is an artificial neural network or a multilayered neural network. The intelligent algorithm may in particular be implemented on a learning and/or determination unit. It is conceivable that the intelligent algorithm is trained using a learning unit and run using a determination unit. The learning unit and the determination unit may in this case be separate from one another. However, it is likewise conceivable that the intelligent algorithm is implemented on a combined learning and determination unit that is formed both to train and to run the intelligent algorithm. For example, the training comprises supervised learning. In this case, training data such as the information input, an imaging parameter and/or an imaging sequence of an imaging examination, a parameter of a workflow of the imaging examination and a desired output such as the determined adaptation of the parameter can be forwarded to the learning and/or determination unit with the artificial neural network. By comparing a target output and an actual output it is possible, in dependence on mathematical methods such as a delta rule, a backpropagation method or an SGD (stochastic gradient descent) method, to draw conclusions of a change to be made to a configuration of the artificial neural network. In this context, changes to the configuration of the artificial neural network may in particular comprise
development of new connections between neurons,
adaptation of weighting of the neurons,
adaptation of a threshold value of the neurons,
addition or deletion of neurons and/or connections between neurons, and
modification of an activation, a propagation and/or an output function.

The terms mentioned are known to those skilled in the art and will not be explained in more detail here. It goes without saying that in addition to the option of supervised learning, further learning methods such as unsupervised learning or reinforced learning are conceivable. Preferably, in the context of training the artificial or multilayered neural network, the target output is a parameter set with adaptation of a parameter that is determined from a concrete request by the user. It is furthermore conceivable that the target output comprises the adaptation of the parameter that is determined from the user's feedback on the first image or the subsequent image. The learning method can thus be trained continuously, in dependence on information inputs made by the user and determined adaptations to parameters of an imaging examination. As described above, the information inputs may also have patient information, clinical findings and/or information on image properties. It is conceivable that an output of the intelligent algorithm is provided, after a predetermined number of training cycles and/or a predetermined accuracy rate, as a standard sequence for an imaging examination.

As a result of training an intelligent algorithm using information inputs made by a user, an imaging examination can advantageously be adapted to specific requirements of the user. As a result, the efficiency of imaging examinations, in particular at hospitals and/or clinics that specialize in the diagnosis of particular regions of the body, can be advantageously enhanced. In particular, the method according to the disclosure can advantageously make a contribution to inexperienced users learning or benefiting from adaptations learned from experienced users.

According to one embodiment of the method according to the disclosure, training of the intelligent algorithm is activated or deactivated in dependence on an item of user information.

An item of user information may be any desired item of information relating to the user of the imaging apparatus, such as educational status, level of experience, specialization, job title, an identifier of the user, or similar. Preferably, the item of user information is retrieved or received at the imaging examination, such that at the start of the imaging examination the user information is known. It is conceivable that training of the intelligent algorithm in dependence on an information input by the user is carried out only in the case of users having a high level of experience (e.g. expert), a specialization corresponding to the imaging examination (e.g. radiologist) and/or a sufficient educational status. It is further conceivable that training of the intelligent algorithm in dependence on the specialization or identifier (e.g. a name and/or staff number) is only possible for a predetermined selection of imaging examinations. For example, rights in respect of training may be linked to a profile of the user, such that the imaging examination automatically activates or deactivates training of the intelligent algorithm when a user logs onto the imaging apparatus, depending on the rights.

As a result of activating or deactivating training of the intelligent algorithm depending on the user information, it is possible to carry out determination of parameters of the imaging examinations in a manner adapted to specific users, by means of the intelligent algorithm. In this way, the complexity of adaptation of the imaging examination to different requirements of users can be advantageously reduced.

In a further embodiment of the method according to the disclosure, adaptation of the parameter is determined in dependence on the trained intelligent algorithm.

It is conceivable that the intelligent algorithm is trained to a frequently used adaptation of a parameter by a user. In one example, the adaptation of the parameter may be determined in dependence on feedback from the user on an image using the trained intelligent algorithm instead of an iterative method. As a result, the time taken for determining the adaptation of the parameter may be advantageously reduced. It is likewise conceivable that the intelligent algorithm is trained to determination of an adaptation of a parameter that is performed in dependence on an item of patient information, a clinical finding and/or an image property. For example, the intelligent algorithm may be trained to determine an adaptation of a parameter if the first image or the subsequent image has a low signal-to-noise ratio, a low resolution and/or an image artifact.

As a result of automatically determining the adaptation of a parameter in dependence on information inputs of this kind, it is possible to advantageously speed up a workflow of the imaging examination. Further, less experienced users can be alerted to adaptations of parameters that have been made by experts. As a result, the quality of the imaging examination and/or the training of members of staff can be advantageously improved.

In one embodiment, the method according to the disclosure has the following step:
carrying out the imaging examination for the purpose of capturing a diagnostic image of a patient,
wherein the parameter of the imaging examination is changed in accordance with the determined adaptation, and wherein the imaging examination is carried out with the changed parameter.

A diagnostic image may be for example a magnetic resonance image, a computed tomography image, an X-ray image or similar of a diagnostically relevant region of the patient's body. Preferably, the imaging examination is carried out with an adapted parameter set that is determined in dependence on the information input according to an embodiment of the method according to the disclosure. The parameter set of the imaging examination thus comprises at least one parameter that has been changed in dependence on an information input. The information input may in this case be performed at the start of the imaging examination, during preparation for the imaging examination and/or during carrying out of the imaging examination.

As a result of carrying out the imaging examination with the adapted parameter set, the imaging examination can be adapted to an information input in a time-efficient and uncomplicated manner. As a result, in a situation that was not predicted such as the discovery of a pathological structure and/or if the image is of low quality, it is advantageously possible to adapt parameters appropriately and promptly and to reduce the risk of having to repeat the imaging examination.

The imaging apparatus according to the disclosure comprises a computing unit that is formed to coordinate a method according to the disclosure and to carry it out using the imaging apparatus.

For the purpose of capturing, processing and storing data such as imaging parameters, parameters of the workflow of the imaging examination, information inputs, information relating to the adaptation of the imaging examination, images, speech messages, machine-readable data and/or data in machine-readable file formats and similar, the imaging apparatus can have further components besides the computing unit. For example, the imaging apparatus can comprise a control unit, a random access memory, a data memory and a suitable interface for the input and output of data. The computing unit may for example comprise a controller, a microcontroller, a CPU, a GPU or similar. The random access memory and the data memory may have storage technologies such as RAM, ROM, PROM, EPROM, EEPROM, flash memory or indeed HDD storage, SSD storage or similar. It is conceivable that the data memory is an internal database that is electrically and/or mechanically connected to the computing unit of the imaging apparatus. However, it is likewise conceivable that the data memory is an external database that is connected to the computing unit by means of a network connection. Examples of external storage units are network servers with corresponding data memories, and a storage unit of a cloud. The data may be transmitted by means of analog and/or digital signals and suitable wired and/or wireless signaling connections between the components of the imaging apparatus. For the purpose of capturing and processing speech messages and/or an interaction with a user, the imaging apparatus may have in particular a speech input unit, a speech processing unit and/or an output unit as further components.

The computing unit and/or the speech processing unit are preferably electrically connected to a control unit of the imaging apparatus and/or integrated in the control unit. The control unit may be formed to carry out a method according to the disclosure, coordinated by the computing unit. The control unit may in particular be formed to carry out an imaging examination of a patient, capture diagnostic images of the patient and transmit the images to other components such as the computing unit, the storage unit and/or an output unit. Furthermore, the control unit may be formed to adapt parameters of the imaging examination, coordinated by the computing unit and/or the speech processing unit.

The computing unit may be formed to determine an item of information relating to an adaptation of the imaging examination in dependence on the information input. Furthermore, the computing unit may be formed to allocate the item of information relating to the adaptation of the imaging examination to a parameter of the imaging examination. Preferably, the computing unit is likewise formed to determine an adaptation of the parameter of the imaging examination in dependence on the information input. The computing unit can furthermore be formed to process feedback from a user on a captured image of the imaging examination. A speech input by the user is preferably captured by means of a speech input unit of the imaging apparatus. In the event of a speech input by the user, the information relating to the adaptation of the imaging examination can in particular be determined by means of the speech processing unit. The speech processing unit may in this case be a discrete component of the imaging apparatus or be integrated into the imaging apparatus.

The components of the imaging apparatus according to the disclosure may advantageously be matched to one another such that it becomes possible to carry out a method according to the disclosure time-efficiently and robustly. In particular, the imaging apparatus according to the disclosure may be formed to coordinate and carry out a workflow of individual method steps autonomously. In this way, adaptation of a parameter of the imaging examination in dependence on the information input can advantageously be determined automatically and/or without any specialized technical knowledge on the part of the user.

The computer program product according to the disclosure is directly loadable into a data memory of a computing unit of an imaging apparatus according to the disclosure, having program code means in order to carry out a method according to the disclosure when the computer program product is executed in the computing unit of the imaging apparatus.

As a result of the computer program product according to the disclosure, the method according to the disclosure can be performed rapidly, identically reproducibly and robustly. The computer program product is configured such that it can perform the method steps according to the disclosure by means of the computing unit. Here, the computing unit must have each of the prerequisites, such as an appropriate random access memory, an appropriate graphics card or an appropriate logic unit, so that the respective method steps can be carried out efficiently. The computer program product is stored for example on a computer-readable medium, or is stored on a network, server or cloud from which it can be loaded into the processor of a local computing unit. The computing unit can in this case take the form of a discrete system component or a part of the imaging apparatus. Furthermore, control information of the computer program product may be stored on an electronically readable data carrier. The control information of the electronically readable data carrier may be formed such that, when the data carrier is used in the computing unit of the imaging apparatus, the control information carries out a method according to the disclosure. Examples of electronically readable data carriers are a DVD, a magnetic tape, a USB stick or any other desired data memories on which electronically readable control information, in particular software, is stored. When this control information is read from the data carrier and transferred to a control unit and/or the computing unit of the imaging apparatus, all the inventive embodiments of the described method according to the disclosure can be carried out.

### BRIEF DESCRIPTION OF THE DRAWINGS

Further advantages and details of the present disclosure will be apparent from the exemplary embodiments described below, and from the drawings, in which:

FIG. 1 shows a schematic representation of an embodiment of an imaging apparatus according to the disclosure,

FIG. 2 shows a flow chart of an embodiment of a method according to the disclosure, and

FIG. 3 shows a flow chart of an embodiment of a method according to the disclosure.

### DETAILED DESCRIPTION

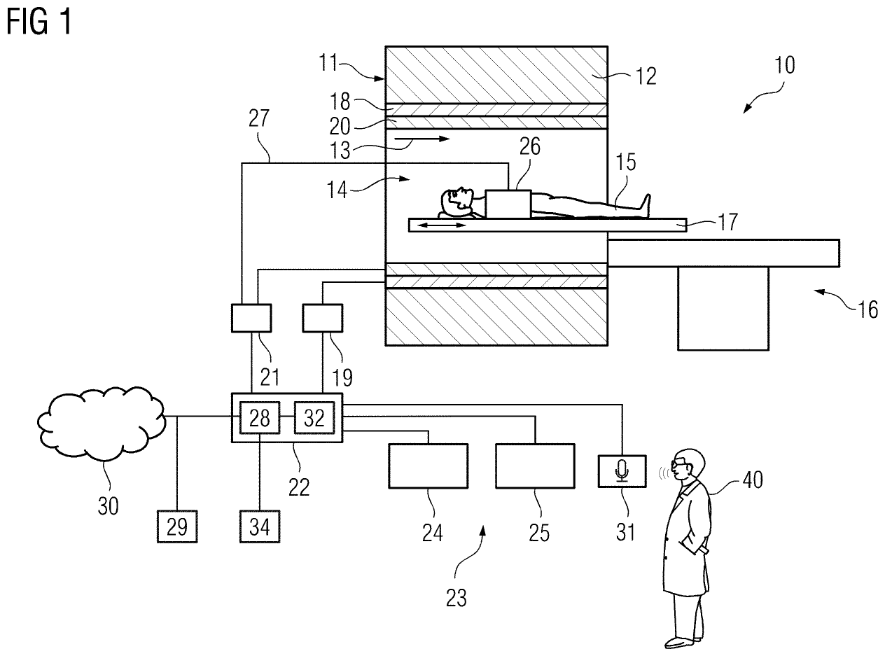

FIG. 1 shows an imaging apparatus according to the disclosure that, in the present example, takes the form of a magnetic resonance apparatus 10. The magnetic resonance apparatus 10 comprises a magnet unit 11, which has for example a permanent magnet, an electromagnet or a superconducting main magnet 12 for generating a strong and in particular homogeneous main magnetic field 13. Moreover, the magnetic resonance apparatus 10 comprises a patient-receiving region 14 for receiving a patient 15. In the present exemplary embodiment, the patient-receiving region 14 is cylindrical in form and is surrounded in a peripheral direction by the magnet unit 11. However, configurations of the patient-receiving region 14 that differ from this example are in principle also conceivable.

The patient 15 can be positioned in the patient-receiving region 14 by means of a patient-positioning apparatus 16 of the magnetic resonance apparatus 10. For this purpose, the patient-positioning apparatus 16 has a patient table 17 that is configured to be movable within the patient-receiving region 14. Furthermore, the magnet unit 11 has a gradient coil 18 for generating magnetic gradient fields that are used for spatial encoding during imaging. The gradient coil 18 is controlled by means of a gradient control unit 19 of the magnetic resonance apparatus 10. Furthermore, the magnet unit 11 may comprise a radio-frequency antenna that, in the present exemplary embodiment, takes the form of a body coil 20 that is permanently integrated in the magnetic resonance apparatus 10. The body coil 20 is formed to excite nuclear spins in the main magnetic field 13 generated by the main magnet 12. The body coil 20 is controlled by a radio-frequency unit 21 of the magnetic resonance apparatus 10, and irradiates an image capture region, substantially formed by a patient-receiving region 14 of the magnetic resonance apparatus 10, with radio-frequency excitation pulses. The body coil 20 may furthermore also be formed to receive magnetic resonance signals.

For the purpose of controlling the main magnet 12, the gradient control unit 19 and for controlling the radio-frequency unit 21, the magnetic resonance apparatus 10 has a control unit 22. The control unit 22 is formed to control the performance of a sequence, such as an imaging GRE (gradient echo) sequence, a TSE (turbo spin echo) sequence or a UTE (ultra-short echo time) sequence. Moreover, the control unit 22 comprises a computing unit 28 for evaluating magnetic resonance data captured during a magnetic resonance examination. The computing unit 28 of the magnetic resonance apparatus 10 may be formed to employ reconstruction methods in order to reconstruct magnetic resonance images using the magnetic resonance data.

The magnetic resonance apparatus 10 may further have a local receiving antenna 26, which is positioned at a diagnostically relevant body region of the patient 15 and detects magnetic resonance signals of the body region of the patient 15 and transmits them to the computing unit 28 of the control unit 22. The local receiving antenna 26 preferably has an electrical terminal line 27 that provides a signaling connection to the radio-frequency unit 21 and the control unit 22. Like the body coil 20, the local receiving antenna 26 may also be formed to excite nuclear spins and to receive magnetic resonance signals. The local receiving antenna 26 may for this purpose be controlled by the radio-frequency unit 21.

Furthermore, the magnetic resonance apparatus 10 comprises a user interface 23, which provides a signaling connection to the control unit 22. Control information such as imaging parameters, but also reconstructed magnetic resonance images, may be displayed to a user 40 at a display unit 24, for example at least one monitor of the user interface 23. Furthermore, the user interface 23 has an input unit 25 by means of which parameters of a magnetic resonance examination can be input by the user 40. In particular, it is conceivable that the user interface 23 has a speech input unit 31 that is formed to capture a speech input of the user 40. The user interface 23 may have for example a desktop computer or take the form of a mobile device such as a smartphone or a tablet computer.

The computing unit 28 and/or the speech processing unit 32 may be formed to receive and process a speech message of the user 40 from the speech input unit 31. In this case, the computing unit 28 may comprise the speech processing unit 32 or be connected to the speech processing unit 32 (see FIG. 1). Further, the computing unit 28 may be formed to output a determined adaptation of a parameter to the user 40 of the magnetic resonance apparatus 10 by way of the display unit 24, and/or to store it in a storage unit 29 and/or a storage unit in a cloud 30. It is furthermore conceivable that the computing unit 28 is formed to transmit the determined adaptation of the parameter to a further magnetic resonance apparatus (not shown) over a network connection or similar. In the present example, the information input is a speech message of the user 40 that is captured by means of the speech input unit 31. The speech input unit 31 may have for example a microphone and/or a sound sensor in order to receive the speech message of the user 40. The magnetic resonance apparatus 10 may further have an output unit (not shown) that, by means of an acoustic output, informs the user 40 of the fact that a change to a parameter of an imaging examination has been determined. However, it is likewise conceivable that the determined adaptation of the parameter is provided to the user 40 by means of the user interface 23, such as a screen with a speaker.

It is furthermore conceivable that the storage unit 29 and/or the storage unit in the cloud 30 have a database with explanations and/or descriptions of parameters of a magnetic resonance examination. The computing unit 28 and/or the speech processing unit 32 may accordingly be formed to access the database in the context of determining the item of information relating to the adaptation of the magnetic resonance examination, in dependence on a speech message of the user 40. For example, the explanations and/or descriptions of the parameters may be used for a semantic search, a keyword search and/or a text mining method. It is likewise conceivable that the item of information relating to adaptation of the magnetic resonance examination is allocated to a parameter of the magnetic resonance examination by means of a processor in the cloud 30 that has access to a search engine and the worldwide web. The processor in the cloud 30 may further have an intelligent algorithm that is trained by the user 40, using the item of information relating to the adaptation of the magnetic resonance examination, and/or is formed to determine the adaptation of the parameter of the magnetic resonance examination in dependence on the information input. Preferably, however, the magnetic resonance apparatus 10 comprises a learning and/or determination unit 34 at which an intelligent algorithm is implemented. The learning and/or determination unit 34 may be directly connected to the computing unit 28 and/or the speech processing unit 32. During training of the intelligent algorithm, the learning and/or determination unit 34 may receive an item of information on the imaging examination and an information input from the computing unit 28. It is possible, after a sufficient number of training cycles, for the learning and/or determination unit 34 to be formed to independently determine an adaptation of a parameter in dependence on the magnetic resonance examination and an information input.

It goes without saying that the illustrated magnetic resonance apparatus 10 may comprise further components that magnetic resonance apparatuses conventionally have. Further, it is also possible for other imaging apparatus such as computed tomography devices, X-ray devices, mammography devices, or similar to have a computing unit 28 that is formed to perform a method according to the disclosure having the imaging apparatus.

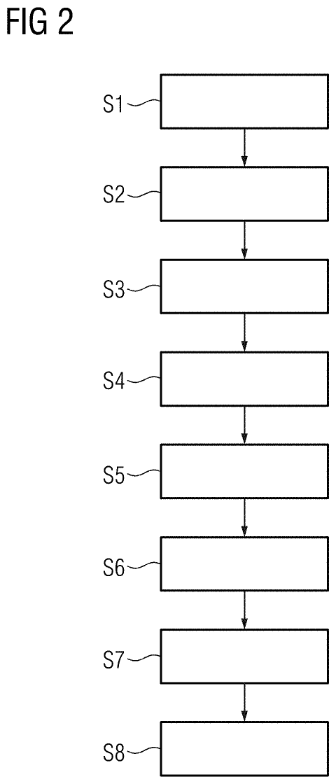

FIG. 2 shows a possible flow chart of a method according to the disclosure for determining an adaptation of a parameter of a magnetic resonance examination that is to be carried out in dependence on an information input for the magnetic resonance examination.

In an optional step S1, a captured magnetic resonance image of the magnetic resonance examination is output to the user 40 of the magnetic resonance examination 10, wherein capture of the information input comprises capture of a feedback message from the user 40 on the captured magnetic resonance image of the magnetic resonance examination. The magnetic resonance image may be output by means of a display unit 24, which preferably has a screen for displaying the magnetic resonance image. The magnetic resonance image may be a first magnetic resonance image of a series of magnetic resonance images that are captured during the magnetic resonance examination. However, it is likewise conceivable that the magnetic resonance image is a subsequent magnetic resonance image. When the magnetic resonance image is output, the user 40 of the magnetic resonance apparatus may be requested to deliver feedback on the magnetic resonance image. Feedback from the user 40 may comprise for example a speech message, a gesture and/or an input by means of any desired input device (e.g. mouse, keyboard) of the user interface 23. Preferably, feedback from the user 40 comprises an item of information relating to an adaptation of the imaging examination, such as an assessment an image quality of the magnetic resonance image and/or a request for adaptation of an imaging parameter, an imaging sequence and/or a parameter of the workflow of the magnetic resonance examination.

In a step S2, an information input for the magnetic resonance examination is captured. Information inputs made by the user 40 may comprise for example an input by means of the input unit 25, a speech input by means of the speech input unit 31, and/or feedback from the user 40 on a magnetic resonance image. Likewise, the feedback from the user 40 may similarly be captured by means of the input unit 25 and/or the speech input unit 31. Moreover, the information input may also be a gesture by the user 40 that is captured by means of a camera in an examination room of the magnetic resonance apparatus 10.

However, the information input may also be performed independently of the user 40. For example, the information input may be an item of patient information on the patient 15, a clinical finding on the patient 15 and/or an item of information on an image property of the magnetic resonance image. Preferably, the computing unit 28 is formed to derive such information inputs in dependence on data from a radiological information system, a hospital information system, a patient registration, a captured magnetic resonance image or similar. The information on the image property may in this case comprise in particular a quantification of a signal-to-noise ratio of the magnetic resonance image and/or an assessment of the presence of image artifacts. For this, the magnetic resonance apparatus 10 may have a dedicated image processing unit that is formed to determine an image property of the magnetic resonance image.

Further, capture of the information input may comprise processing of the information input. For this purpose, a signal of the information input such as a noise signal, a speech message, a camera signal, a text-based message or similar is preferably converted into an electrical signal and/or into machine-readable data. Machine-readable data may take the form among other things of binary code, hexadecimal numbers, a high-level language and be in suitable file formats such as RDFa, HTML, CSV, XML and similar. For example, a speech input by the user 40 is captured using the speech input unit 31 and converted into machine-readable data. Then, the speech input may be processed using the computing unit 28 and/or the speech processing unit 32. Here, speech processing may have one or more of the following steps, which are known to those skilled in the art:
speech recognition,
tokenization,
morphological analysis,
syntactical analysis,
semantic analysis, and
dialog analysis.

In one embodiment, the speech input is processed using an artificial neural network or a multilayered neural network (e.g. MultiNet). However, it is likewise conceivable that an individual one or a plurality of the listed steps of speech processing are performed using an artificial neural network or a multilayered neural network.

According to a further embodiment, processing of the speech input is performed using a statistical model and/or a logical data model. Preferably, the speech input is processed using a statistical model and/or a logical data model according to individual or all of the above-listed steps of speech processing. It is conceivable that, when corresponding models are used, the user 40 makes use of a predetermined selection of terms or instructions, wherein this selection is matched to the imaging apparatus and/or the speech input unit.

A method for speech processing carried out using the computing unit 28 and/or the speech processing unit 32. However, it is likewise conceivable that the method for speech processing is implemented on an external processor, in particular a processor in the cloud 30. In this case, the speech input can be transmitted to the processor in the cloud 30, and this processor processes the speech input and sends an item of information relating to the adaptation of the magnetic resonance implementation back to the computing unit 28.

In a further step S3, an item of information relating to an adaptation of the magnetic resonance examination is determined in dependence on the information input. The item of information relating to adaptation of the magnetic resonance examination may be determined in a complex manner from processed information inputs made by the user 40 and from an item of patient information, a clinical finding and/or an item of information on an image property. Here, for each information input a check may be carried out of the information input in respect of correlation with an imaging parameter, an imaging sequence and/or a workflow of the magnetic resonance examination. Preferably, the item of information relating to the adaptation of the magnetic resonance examination comprises an indication of the direction in which the adaptation of a parameter is to be made. While it is possible for a user 40 to input a desired change to an imaging parameter using a speech message, an indication of the direction in which the adaptation of a parameter is to be made may, within the context of the method according to the disclosure, also be determined automatically in dependence on an image property of a magnetic resonance image. Undesired image properties may be for example a low signal-to-noise ratio and/or or an undesired position of a diagnostic relevant region of the body of the patient 15 in an imaging region.

In a further step S4, the item of information relating to the adaptation of the magnetic resonance examination is allocated to a parameter of the magnetic resonance examination. Here, allocation is preferably performed by means of a classification. It is conceivable that the classification comprises use of a neural network, a multilayered neural network and/or a text mining method. It is also possible for the classification to comprise formation of tuples, vectors, matrices and/or any desired data structure, wherein these allocate the item of information on the change to the imaging examination to a parameter of the imaging examination. It is furthermore conceivable that allocation of the item of information relating to the adaptation of the imaging examination is performed using a model, such as a statistical model and/or a logical data model. The method for classifying the item of information relating to the adaptation of the magnetic resonance examination may be implemented for example at the computing unit 28 and/or a processor in the cloud 30.

In a further step S5, an adaptation of a parameter of the magnetic resonance examination is determined in dependence on the information input. Preferably, the adaptation of a parameter is determined using the item of information relating to the adaptation of the magnetic resonance examination. For example, the item of information relating to the adaptation of the magnetic resonance examination may comprise a desire of the user 40 in respect of a lower contrast of fatty tissue in a magnetic resonance image. Here, determination of the adaptation in relation to relevant imaging parameters, such as a time to echo and/or a repetition time, may be performed taking into account a T1 relaxation time and/or a T2 relaxation time of fatty tissue. Corresponding data on the fatty tissue is preferably stored in and retrievable from the storage unit 29 or a storage unit in the cloud 30.

In a preferred embodiment, the adaptation of the parameter is determined in dependence on the speech input made by the user 40. Preferably, the item of information relating to the adaptation of the magnetic resonance examination is determined by means of one of the above-described methods for processing the speech input. It is furthermore conceivable that the computing unit 28 is formed to prioritize different information inputs, in dependence on the items of information relating to the adaptation of the magnetic resonance examination. In this way, from a plurality of information inputs it is possible to draw on an information input having top priority, such as the safety of the patient 15 in relation to a specific absorption rate (or a radiation dose in X-ray imaging), for the purpose of determining the adaptation of the parameter. However, it is also possible for the computing unit 28 to be formed to determine a compromise between a plurality of information inputs. In this case, a compromise of the parameters may be found using the plurality of conflicting information inputs, such as a high number of magnetic resonance images and the taking into account of the specific absorption rate.

In an optional step S6, an intelligent algorithm is trained with respect to determining adaptation of the parameter, wherein the intelligent algorithm is trained at least in dependence on the information input, the magnetic resonance examination to be carried out, and the determined adaptation of the parameter. Preferably, training of the intelligent algorithm comprises supervised learning of a multilayered neural network. The multilayered neural network may in this case have for example two layers (hidden layers), three layers, four layers or more than four layers. During the training, for each magnetic resonance examination in which an adaptation of a parameter in dependence on an information input is determined, a training data set may be transmitted to the learning and/or determination unit 34 on which the multilayered neural network is implemented. The training data set preferably comprises at least information relating to the magnetic resonance examination, the information input and the adaptation of the parameter that has been determined in dependence on the information input. The item of information relating to the magnetic resonance examination may for example comprise a parameter set of a standard sequence that prevailed before adaptation of the parameter was determined.

In one example, the multilayered neural network is trained by a supervised learning method. Here, the parameter set of the magnetic resonance examination having the changed parameter that was determined in dependence on the information input may be a target output of the multilayered neural network. The target output can be compared with an actual output that the multilayered neural network generates in the current state. For this purpose, an input pattern such as the standard sequence and/or the information input may be propagated forward by the multilayered neural network. It is conceivable that an item of information relating to the adaptation of the magnetic resonance examination in dependence on the information input has already been determined by the computing unit 28 and/or the speech processing unit 32 before it is transmitted as an information input to the learning and/or determination unit 34. In the course of the training procedure, the information input (or the item of information relating to the adaptation of the magnetic resonance examination) may be regarded for example as a parameter of the parameter set of the magnetic resonance examination, or implemented as a separate input variable, such as a label of the input pattern. Furthermore, it goes without saying that further possible implementations are conceivable.

The multilayered neural network may be trained by comparing the actual output and the target output. It is conceivable that a backpropagation method or an SGD (stochastic gradient descent) method is used for this purpose. For example, in the backpropagation method a difference between the actual output and the target output of the multilayered neural network is formed, and this difference is considered an error. The error can then be backpropagated from an output layer to an input layer of the multilayered neural network. Here, a configuration of the multilayered neural network, in particular a weighting of connections between neurons, can be changed depending on its effect on the error. By means of corresponding methods, the error between the target output and the actual output of the multilayered neural network for an input pattern can be minimized. It is also conceivable, referring to FIG. 1, that the learning and/or determination unit 34 is implemented on a processor in the cloud 30. In this way, the learning and/or determination unit 34 may be trained by a multiplicity of data sets of magnetic resonance apparatuses that are connected to the cloud 30.

In one embodiment, rights of a user 40 in respect of training are linked to a profile of the user 40, such that training of the intelligent algorithm is automatically activated or deactivated when a user 40 logs onto the imaging apparatus, depending on the rights.

In a step S7, a parameter set of the magnetic resonance examination is provided, wherein the provided parameter set has a parameter that is changed in accordance with the determined adaptation, and wherein the parameter set is stored in a storage unit that is connected to an imaging apparatus. Provision comprises at least storage of the parameter set of the magnetic resonance examination having the changed parameter, in the storage unit 29 of the magnetic resonance apparatus 10 and/or a storage unit in the cloud 30. It is furthermore conceivable that the parameter set having the changed parameter is transmitted to a further magnetic resonance apparatus. In this way, a user 40 of the magnetic resonance apparatus 10 can also make use of adaptations on a further magnetic resonance apparatus that have been made by means of the method according to the disclosure under particular conditions for a particular magnetic resonance examination. Further, provision of the parameter set may also comprise visual output of the parameter set to the user 40, by means of the display unit 24. Preferably, the changed parameter is indicated by a marker and/or highlighted in the visual output so that the user 40 can quickly ascertain which parameters of the parameter set have been changed.

In an optional step S8, the magnetic resonance examination is carried out for the purpose of capturing a diagnostic image of a patient, wherein the parameter of the magnetic resonance examination is changed in accordance with the determined adaptation, and wherein the magnetic resonance examination is carried out using the changed parameter. It is conceivable that an information input is captured even before capture of the first magnetic resonance image, for example during preparation of the magnetic resonance examination and/or during patient registration. In this case, the magnetic resonance examination may be started up using the changed parameter. However, it is likewise conceivable that the information input comprises feedback from the user 40 on the first magnetic resonance image, the subsequent magnetic resonance image and/or a clinical finding that is derived in dependence on a magnetic resonance image. In such cases, the magnetic resonance examination may be propagated using the parameter that has been changed in dependence on the feedback from the user 40 and/or the clinical finding.

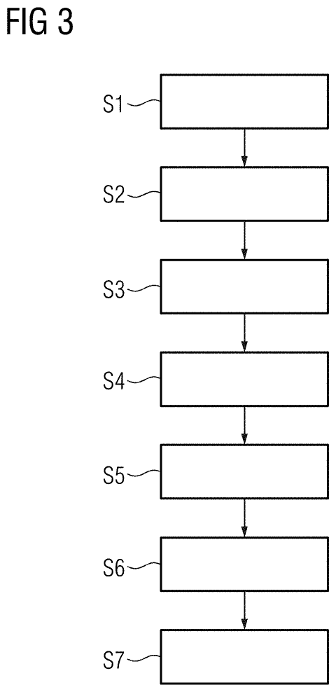

FIG. 3 shows a flow chart of a further embodiment of the method according to the disclosure. In this embodiment, the steps S1 to S7 as described above may be carried out at a first point in time. The first point in time is in particular characterized in that an intelligent algorithm of a learning and/or determination unit 34 such as a multilayered neural network is trained in dependence on a magnetic resonance examination, an information input and a determined adaptation of a parameter.

At a second point in time, the intelligent algorithm has completed a sufficient number of training cycles. It is conceivable that the step S5 of determining the adaptation of the parameter of the magnetic resonance examination in dependence on the information input is performed using the intelligent algorithm from the second point in time onward. In one embodiment, the computing unit 28 is formed to retrieve the determination of the adaptation of a parameter from the learning and/or determination unit 34 at the second point in time. For this purpose, the learning and/or determination unit 34 may for example receive a parameter set of the magnetic resonance examination and an item of information relating to adaptation of the magnetic resonance examination, and provide a parameter set having at least one changed parameter. The step S4 of allocating the item of information relating to adaptation of the magnetic resonance examination to a parameter of the magnetic resonance examination may be implicitly performed using the intelligent algorithm, at the second point in time. It is likewise conceivable that the step S6 of training the intelligent algorithm in respect of determining the adaptation of the parameter is dispensed with from the second point in time onward. Preferably, however, the intelligent algorithm may run through further training cycles, for example if, during step S3 of determining an item of information relating to adaptation of the magnetic resonance examination, a request from a user 40 with expert specialist knowledge for a change to a parameter is determined. It is furthermore conceivable that training of the intelligent algorithm and/or use of the intelligent algorithm for determining an adaptation of a parameter may be activated or deactivated manually for a predetermined magnetic resonance examination and/or as a global setting of the magnetic resonance apparatus 10.

As described above, the computing unit 28 may further be formed to carry out the magnetic resonance examination using the parameter set having the changed parameter. It is also conceivable that at the second point in time the parameter set of the magnetic resonance examination is the current one and/or that there is no information input. In this case, the computing unit 28 may continue with coordination of the magnetic resonance examination without any change to a parameter. In a further embodiment, the intelligent algorithm may likewise be trained at the second point in time to determine (S3) the item of information relating to adaptation of the magnetic resonance examination in dependence on the information input according to one of the above-described methods, in particular using an artificial neural network or a multilayered neural network.

It goes without saying that the embodiments of the inventive method and the inventive magnetic resonance apparatus that are described here should be understood as exemplary. Individual embodiments can thus be extended by features of other embodiments. In particular, the order in which the method steps of the inventive method are performed should be understood as exemplary. The individual steps may also be carried out in a different order, or may overlap one another in time, partly or completely.

1. A computer-implemented method for determining an adaptation of a parameter of an imaging examination that is to be carried out using a medical imaging apparatus in dependence on an input of information for the imaging examination, comprising:
capturing the information input for the imaging examination;
determining an item of information relating to an adaptation of the imaging examination in dependence on the information input;
allocating the item of information relating to the adaptation of the imaging examination to a parameter of the imaging examination;
determining an adaptation of the parameter of the imaging examination in dependence on the information input; and
providing a parameter set of the imaging examination, wherein the provided parameter set has a parameter that is changed in accordance with the determined adaptation, and wherein the parameter set is stored in storage that is connected to an imaging apparatus.

2. The method as claimed in claim 1, wherein the parameter of the imaging examination comprises an imaging parameter, an imaging sequence, and/or a parameter of a workflow of the imaging examination.

3. The method as claimed in claim 1, wherein the information input comprises a speech input from a user, the capturing of the information input comprises processing of the speech input from the user, and the adaptation of the parameter is determined in dependence on the speech input from the user.

4. The method as claimed in claim 3, wherein the speech input from the user is processed by means of an artificial neural network or a multilayered neural network.

5. The method as claimed in claim 3, wherein the speech input from the user is processed by means of a statistical model and/or a logical data model.

6. The method as claimed in claim 1, further comprising:
outputting a captured image of the imaging examination to the user of the imaging apparatus,
wherein the capturing of the information input comprises capturing feedback from the user on the captured image of the imaging examination, and the adaptation of the parameter is determined in dependence on the feedback from the user.

7. The method as claimed in claim 6, wherein the capturing of the information input comprises capturing feedback from the user on an imaging parameter and/or an image property.

8. The method as claimed in claim 1, wherein the information input comprises at least:
an input from the user,
a speech input from the user,
a feedback message from the user on a captured image of the imaging examination,
an item of information on a patient,
a clinical finding, and/or
an item of information on an image property.

9. The method as claimed in claim 1, further comprising:
training an intelligent algorithm in respect of determining an adaptation of a parameter,
wherein the intelligent algorithm is trained at least in dependence on the information input, the imaging examination to be carried out, and the determined adaptation of the parameter.

10. The method as claimed in claim 9, wherein the intelligent algorithm is an artificial neural network or a multilayered neural network.

11. The method as claimed in claim 9, wherein the training of the intelligent algorithm is activated or deactivated in dependence on an item of user information.

12. The method as claimed in claim 9, wherein the adaptation of the parameter is determined in dependence on the trained intelligent algorithm.

13. The method as claimed in claim 1, further comprising:
carrying out the imaging examination for the purpose of capturing a diagnostic image of a patient,
wherein the parameter of the imaging examination is changed in accordance with the determined adaptation, and the imaging examination is carried out with the changed parameter.

14. An imaging apparatus, comprising:
a computer formed to coordinate a method as claimed in claim 1 and to carry the method out using the imaging apparatus.

15. A non-transitory computer program product, which is directly loadable into a data memory of a computer of an imaging apparatus, having program code to carry out the method as claimed in claim 1 when the computer program product is executed in the computer of the imaging apparatus.
